(19) United States
(12) Patent Application Publication
(10) Pub. No.: US 2025/0051804 A1
(43) Pub. Date: Feb. 13, 2025

(54) Method to Transform Phosphorus in an Antibiotic into Sulfur

(71) Applicant: Bradley Zang, Jacksonville, FL (US)

(72) Inventor: Bradley Zang, Jacksonville, FL (US)

(21) Appl. No.: 18/232,111

(22) Filed: Aug. 9, 2023

(51) Int. Cl. C12P 3/00

(52) U.S. Cl. CPC C12P 3/00

(57) ABSTRACT

Certain bacteria, which are used to develop antibiotics, is very beneficial to humans in the control of diseases or other ailments and phosphorous is a common element in many of those bacteria/antibiotic formulations. However, phosphorous is contraindicated at times during the treatment of ailments, particularly those with liver or kidney ailments. One of those ailments is patients who are undergoing dialysis treatment. This is a process that will naturally and safely remove phosphorus from the bacteria compound and replace it with sulfur, which allows the bacteria to maintain its chemical bonds while retaining the beneficial effect of the treatment.

### PRIORITY REFERENCE

This application seeks the priority date from a previously filed provisional application with a filing date of Aug. 9, 2022 and associated Ser. No. 63/396,462.

### BACKGROUND OF THE INVENTION

### A. Field of the Invention

This relates to removing elements from certain chemical compounds and specifically in the manufacture of antibiotics. Certain elements of certain compounds may be counter-productive or contraindicated in certain patients. For instance, dialysis patients should not be exposed to phosphorus because phosphorus will counteract the effects of the dialysis and may harm additional to the patient. With this process the phosphorous is removed and transformed into sulfur while maintaining the original compound.

### B. Prior Art

There are many prior art references the teach the use of radioactive devices to achieve certain desired results. A representative example of this type of device can be found at Schwartz U.S. Pat. No. 8,993,117, which teaches the use of radioactive material in a medical instrument. Another example can be found at Sidheswaran, U.S. Pat. No. 10,150,100 which teaches a method to remove a particle using carbon fibers. Another example can be found at Lazarus, US Patent Publication 20024/004837.

None of the prior art references use an isotope of an element to transform an existing element into a different element in a way that is described by this application.

### BRIEF SUMMARY OF THE INVENTION

This is a method to transform an element in a compound—specifically changing phosphorus to sulfur in a chemical compound and more specifically a bacteria that produces an antibiotic. Some bacteria produce antibiotics that contain phosphorus and phosphorus is critical in many compounds. Phosphorus, however, is contraindicated in individuals who suffer from liver or kidney problems. For instance, dialysis patients should avoid excessive amounts of phosphorus.

There are certain well-known chemical compounds, such as fosfazinomycin and moenomyein A. Both these compounds have phosphorus elements in the chemical chain that need to be removed and should be replaced with sulfur elements, which tend to have less detrimental effects for the patient. During the process, the bacteria is grown with plasmids to increase production and normal growth medium; this type of growth is very common and is not novel to this application.

In order to transform phosphorus into sulfur naturally a radioactive phosphorus isotope, which is an element that has the same protons but different neutrons, is introduced into the antibiotic. The isotope will have a free electron and the free electron effects the transmutation of the phosphorus into sulfur.

The radioactive isotope has an electron which will break away from the phosphorus and naturally transform phosphorus into sulfur. This would transform the phosphorus to sulfur and, therefore, make a different antibiotic treatment for use in other applications that can be tolerated by many more patients.

### BRIEF DESCRIPTION OF THE DRAWINGS

There is no requirement for drawings related to this process.

### DETAILED DESCRIPTION OF THE EMBODIMENTS

This is a method to transform an element in a compound—specifically changing phosphorus to sulfur in a chemical compound and more specifically a bacteria that produces an antibiotic. Some bacteria produce antibiotics that contain phosphorus and phosphorus is critical in many compounds. Phosphorus, however, is contraindicated in individuals who suffer from liver or kidney problems. For instance, dialysis patients should avoid excessive amounts of phosphorus.

There are certain well-known chemical compounds, such as fosfazinomycin and moenomyein A. Both these compounds have phosphorus elements in the chemical chain that need to be removed and should be replaced with sulfur elements, which tend to have less detrimental effects for the patient. During the process, the bacteria is grown with plasmids to increase production and normal growth medium; this type of growth is very common and is not novel to this application.

In order to transform phosphorous into sulfur naturally a radioactive phosphorus isotope, which is an element that has the same protons but different neutrons, is introduced into the antibiotic. The isotope will have a free electron and the free electron effects the transmutation of the phosphorus into sulfur.

The radioactive isotope of phosphorus is introduced into the bacteria compound. The radioactive isotope has an electron which will break away from the phosphorus and naturally transform phosphorous into sulfur. This would transform the phosphorus to sulfur and, therefore, make a different antibiotic treatment for use in other applications that can be tolerated by many more patients.

Each of the radioactive isotopes has a certain half-life which must be taken into account; while there are different phosphorus isotopes, the selection of a particular isotope may depend on the availability of the isotope or the cost of the isotope. It is also very important that any radioactive material from the isotope be removed. The removal of any radioactive element in the bacteria is essential for the safety of the patient. After the culture is formed and the isotope is introduced the bacteria is lysed (which is common) the bacteria is diluted with water. The bacteria is also lysed or broken down and diluted with water. Dilution is important to make sure that the additional electrons from the phosphorus do not destroy the bacteria.

Cryogenic freezing of the solution with the lysed and diluted compound is now performed. Cryogenic freezing and the radioactive isotope is essential because the decay of the element (phosphorous) through the use of the free electron process takes a very long time and cannot be rushed. For instance, the half-life of the radioactive phosphorous 32 is 14 days and for phosphorus 33 is 25 days. Freezing is essential to prevent the original bacteria from decaying and losing its beneficial properties and is also important to absorb some of the energy from the beta emissions and prevent deterioration of the bacteria.

It is anticipated that the cryogenically frozen solution must be kept for over 200 days where the phosphorus content will be reduced by a million fold and the sulfur replaces the original phosphorus. The very low temperature that is achieved by cryogenic freezing avoids any further changes to the structure of the product or original bacteria.

While the embodiments of the invention have been disclosed, certain modifications may be made by those skilled in the art to modify the invention without departing from the spirit of the invention.

The inventor claims:

1. The method to transform phosphorous in an antibiotic into sulfur which is comprised of the following steps:
growing a culture of bacteria,
monitoring the growth of the bacteria into an antibiotic,
determining the amount of phosphorous in the culture,
introducing a radioactive phosphorous isotope into the culture,
lysing the bacteria,
diluting the bacteria,
cryogenically freezing the bacteria,
storing the cryogenically frozen bacteria,
monitoring the culture.

2. The method to transform phosphorous in an antibiotic into sulfur as described in claim 1 wherein the radioactive phosphorous is phosphorous 32.

3. The method to transform phosphorous in an antibiotic into sulfur as described in claim 1 wherein the radioactive phosphorous is phosphorous 33.

4. The method to transform phosphorous in an antibiotic into sulfur as described in claim 1 wherein the storage is in the range of two hundred and three hundred sixty-five days.
